(19) United States
(12) Patent Application Publication
(10) Pub. No.: US 2025/0369946 A1
(43) Pub. Date: Dec. 4, 2025

(54) METHOD FOR REAL TIME PHYSICS DRIVEN MACHINE LEARNING BASED PREDICTIVE AND PREVENTIVE ADVISORY FOR OIL IN PRODUCED WATER ESTIMATION

(71) Applicant: SAUDI ARABIAN OIL COMPANY, Dhahran (SA)

(72) Inventors: Syed Abrar Ahmed, Dhahran (SA); Ahmad Hussain Al-Zubail, Dhahran (SA)

(73) Assignee: SAUDI ARABIAN OIL COMPANY, Dhahran (SA)

(21) Appl. No.: 18/876,909

(22) PCT Filed: Jun. 30, 2023
(86) PCT No.: PCT/US2023/026704

§ 371 Date: Dec. 19, 2024

Related U.S. Application Data
(60) Provisional application No. 63/357,513, filed on Jun. 30, 2022.

(51) Int. Cl. G01N 33/18; G01N 33/28

(52) U.S. Cl. CPC G01N 33/1833; G01N 33/2823; G01N 33/2847

(57) ABSTRACT

A method to perform oil in produced water analysis allows measuring the large volume of oil in produced water reliably. In the method, a time-series and physics based machine learning model of a gas oil separation plant is generated, advisory actionable items for maintaining a crude oil quality within a pre-determined threshold are generated based on machine learning model coefficients and outputs of soft sensors, and then the advisory actionable items are presented to a user.

### BACKGROUND

In conventional oil fields operations, water quality monitoring in terms of oil in produced water does not receive attention commensurate with their economic and environmental importance. Operations and Engineering in oil trains, often called as GOSPs, rely on sampling data and in some cases on online expensive tools that ignore the complex interplay of flow regimes, chemicals, and emulsions on oil content during crude production. These conventional methods cannot measure the large volume of oil in produced water and uses only a fraction of sample to represent the entire produced water stream.

Machine learning (ML) is a type of artificial intelligence (AI) that allows software applications to become more accurate at predicting outcomes without being explicitly programmed to do so. Machine learning algorithms use historical data as input to predict new output values to train a machine learning model. Mathematically, a feature is an input variable of the machine learning model, and a label is an output variable of the machine learning model. The machine learning model defines the relationship between features and label. The machine learning model is trained by optimizing an objective function (e.g., an error score) using training dataset to find an optimal solution of machine learning model parameters. The objective function is optimized by plugging candidate solutions into a model to find a particular solution with optimal value of the objective function against the training dataset.

In game theory, Shapley values represent the marginal contribution of each player to the end result. When a machine learning model is viewed as a game in which individual features “cooperate” together to produce an output, i.e., a model prediction, Shapley values attribute the prediction to each of the input features. Python is a computer programming language often used to build websites and software, automate tasks, and conduct data analysis. SHAP is a Python library that uses Shapley values to explain the output of any machine learning model. Explainable artificial intelligence (XAI) is a set of processes and methods that allows human users to comprehend and trust the results and output created by machine learning algorithms. Explainable AI is used to describe an AI model, its expected impact and potential biases.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to workflows and methods to perform machine learning based analysis of oil in produced water considering the difficulties mentioned above. These workflows and methods allow measuring the large volume of oil in produced water reliably.

In one or more embodiments, a method to perform oil in produced water analysis includes steps of generating a time-series and physics based machine learning model of a gas oil separation plant; generating, based on machine learning model coefficients and outputs of soft sensors, advisory actionable items for maintaining a crude oil quality within a pre-determined threshold; and presenting, to a user, the advisory actionable items. The step of generating the time-series and physics based machine learning model includes performing feature engineering based on one or more of calculating missing flow rates using mass balance, calculating water concentration of output from a low pressure production trap (LPPT) of the gas oil separation plant, and estimating water fraction out of a dehydrator of the gas oil separation plant. The step of generating the time-series and physics based machine learning model further includes augmenting the time-series and physics based machine learning model with natural language processing inputs from the user.

In one or more embodiments, the method further includes preprocessing and feature reduction, in which the preprocessing comprises identifying process intelligence (PI) tags corresponding to a time series data and a single value over time, preprocessing data, and setting up a machine learning frame work to identify crude quality parameters. Preprocessing data includes removing outliers, transforming binary to binary values, transforming string values to numeric values, removing non-relevant data, and interpolating missing values. The feature reduction includes using the PI tags to retrieve archived big data. The PI tags are sorted to remove and aggregate redundant tags. The advisory actionable items identify root causes for poor water separation.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIGS. 1 and 2 show systems in accordance with one or more embodiments.

FIGS. 3, 4, 5A and 5B show examples in accordance with one or more embodiments.

FIG. 6 shows a computer system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments of the disclosure include systems and methods for a multi-target time-series based hybrid physics based machine learning architecture to evaluate the real time water content in crude oil and its impact on crude production over the operation of the facility, storage and transportation. A decision tree-based algorithm is trained to predict oil in produced water using process parameters. The model predicts oil content in produced water. Data from the other process trains to the model, possessing enough information and production data to be used in the model. The data is split randomly into an 80%/20% training & testing split. In order to understand the model predictions, advanced AI models are employed that reflect how each feature contributes to the oil in water predictions. This approach offers a highly accurate way of predicting oil in produced water and at analyzing the operator choices that impact crude quality.

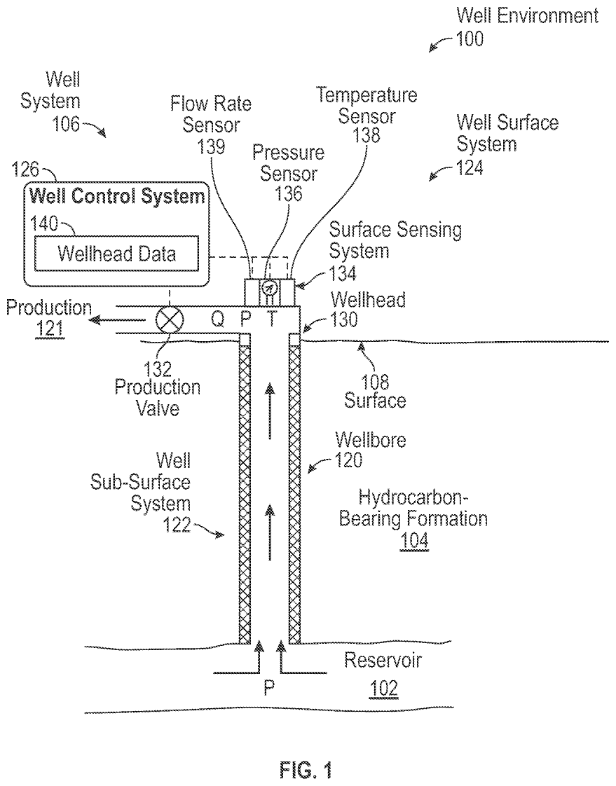

Turning to FIG. 1, FIG. 1 shows a schematic diagram in accordance with one or more embodiments. As shown in FIG. 1, FIG. 1 illustrates a well environment (100) that includes a hydrocarbon reservoir (“reservoir”) (102) located in a subsurface hydrocarbon-bearing formation (104) and a well system (106). The hydrocarbon-bearing formation (104) may include a porous or fractured rock formation that resides underground, beneath the earth's surface (“surface”) (108). In the case of the well system (106) being a hydrocarbon well, the reservoir (102) may include a portion of the hydrocarbon-bearing formation (104). The hydrocarbon-bearing formation (104) and the reservoir (102) may include different layers of rock having varying characteristics, such as varying degrees of permeability, porosity, and resistivity. In the case of the well system (106) being operated as a production well, the well system (106) may facilitate the extraction of hydrocarbons (or “production”) from the reservoir (102).

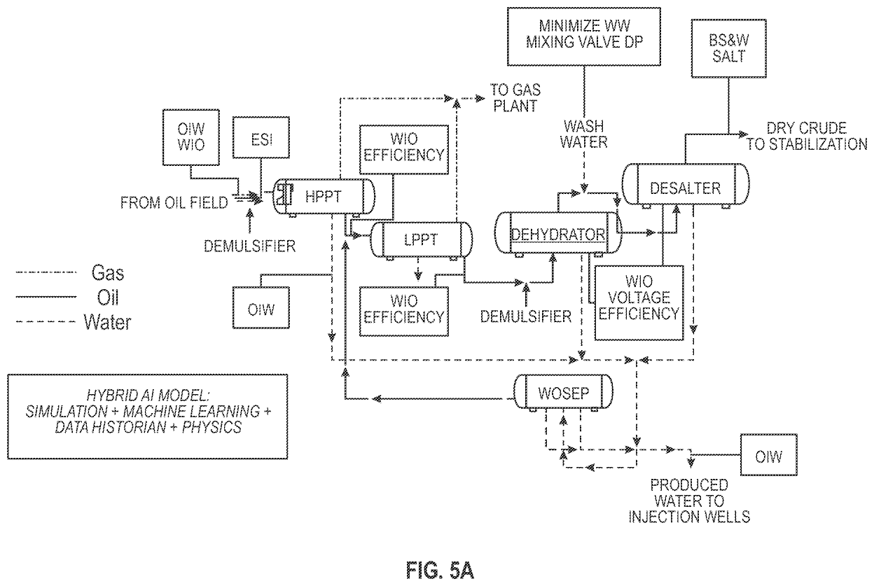

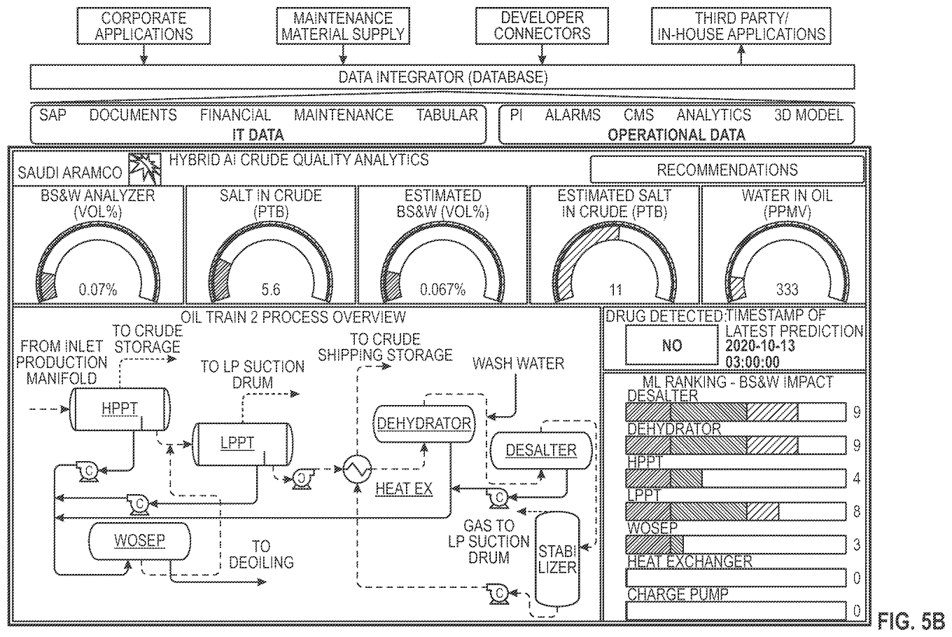

In some embodiments, the well system (106) includes a wellbore (120), a well sub-surface system (122), a well surface system (124), and a well control system (126). The control system (126) may control various operations of the well system (106), such as well production operations, well completion operations, well maintenance operations, and reservoir monitoring, assessment and development operations. In some embodiments, the control system (126) includes a computer system that is the same as or similar to that of computer system (502) described below in FIG. 5 and the accompanying description.

The wellbore (120) may include a bored hole that extends from the surface (108) into a target zone of the hydrocarbon-bearing formation (104), such as the reservoir (102). An upper end of the wellbore (120), terminating at or near the surface (108), may be referred to as the “up-hole” end of the wellbore (120), and a lower end of the wellbore, terminating in the hydrocarbon-bearing formation (104), may be referred to as the “down-hole” end of the wellbore (120). The wellbore (120) may facilitate the circulation of drilling fluids during drilling operations, the flow of hydrocarbon production (“production”) (121) (e.g., oil and gas) from the reservoir (102) to the surface (108) during production operations, the injection of substances (e.g., water) into the hydrocarbon-bearing formation (104) or the reservoir (102) during injection operations, or the communication of monitoring devices (e.g., logging tools) into the hydrocarbon-bearing formation (104) or the reservoir (102) during monitoring operations (e.g., during in situ logging operations).

In some embodiments, during operation of the well system (106), the control system (126) collects and records wellhead data (140) for the well system (106). The wellhead data (140) may include, for example, a record of measurements of wellhead pressure (P) (e.g., including flowing wellhead pressure (FWHP)), wellhead temperature (T) (e.g., including flowing wellhead temperature), wellhead production rate (Q) over some or all of the life of the well (106), and water cut data. In some embodiments, the measurements are recorded in real-time, and are available for review or use within seconds, minutes or hours of the condition being sensed (e.g., the measurements are available within 1 hour of the condition being sensed). In such an embodiment, the wellhead data (140) may be referred to as “real-time” wellhead data (140). Real-time wellhead data (140) may enable an operator of the well (106) to assess a relatively current state of the well system (106), and make real-time decisions regarding development of the well system (106) and the reservoir (102), such as on-demand adjustments in regulation of production flow from the well.

With respect to water cut data, the well system (106) may include one or more water cut sensors. For example, a water cut sensor may be hardware and/or software with functionality for determining the water content in oil, also referred to as “water cut.” Measurements from a water cut sensor may be referred to as water cut data and may describe the ratio of water produced from the wellbore (120) compared to the total volume of liquids produced from the wellbore (120). Water cut sensors may implement various water cut measuring techniques, such as those based on capacitance measurements, Coriolis effect, infrared (IR) spectroscopy, gamma ray spectroscopy, and microwave technology. Water cut data may be obtained during production operations to determine various fluid rates found in production from the well system (106). This water cut data may be used to determine water-to-gas information regarding the wellhead (130).

In some embodiments, a water-to-gas ratio (WGR) is determined using a multiphase flow meter. For example, a multiphase flow meter may use magnetic resonance information to determine the number of hydrogen atoms in a particular fluid flow. Since oil, gas and water all contain hydrogen atoms, a multiphase flow may be measured using magnetic resonance. In particular, a fluid may be magnetized and subsequently excited by radio frequency pulses. The hydrogen atoms may respond to the pulses and emit echoes that are subsequently recorded and analyzed by the multiphase flow meter.

In some embodiments, the well surface system (124) includes a wellhead (130). The wellhead (130) may include a rigid structure installed at the “up-hole” end of the wellbore (120), at or near where the wellbore (120) terminates at the Earth's surface (108). The wellhead (130) may include structures for supporting (or “hanging”) casing and production tubing extending into the wellbore (120). Production (121) may flow through the wellhead (130), after exiting the wellbore (120) and the well sub-surface system (122), including, for example, the casing and the production tubing. In some embodiments, the well surface system (124) includes flow regulating devices that are operable to control the flow of substances into and out of the wellbore (120). For example, the well surface system (124) may include one or more production valves (132) that are operable to control the flow of production (134). For example, a production valve (132) may be fully opened to enable unrestricted flow of production (121) from the wellbore (120), the production valve (132) may be partially opened to partially restrict (or “throttle”) the flow of production (121) from the wellbore (120), and production valve (132) may be fully closed to fully restrict (or “block”) the flow of production (121) from the wellbore (120), and through the well surface system (124).

Keeping with FIG. 1, in some embodiments, the well surface system (124) includes a surface sensing system (134). The surface sensing system (134) may include sensors for sensing characteristics of substances, including production (121), passing through or otherwise located in the well surface system (124). The characteristics may include, for example, pressure, temperature, and flow rate of production (121) flowing through the wellhead (130), or other conduits of the well surface system (124), after exiting the wellbore (120).

In some embodiments, the surface sensing system (134) includes a surface pressure sensor (136) operable to sense the pressure of production (151) flowing through the well surface system (124), after it exits the wellbore (120). The surface pressure sensor (136) may include, for example, a wellhead pressure sensor that senses a pressure of production (121) flowing through or otherwise located in the wellhead (130). In some embodiments, the surface sensing system (134) includes a surface temperature sensor (138) operable to sense the temperature of production (151) flowing through the well surface system (124), after it exits the wellbore (120). The surface temperature sensor (138) may include, for example, a wellhead temperature sensor that senses a temperature of production (121) flowing through or otherwise located in the wellhead (130), referred to as “wellhead temperature” (T). In some embodiments, the surface sensing system (134) includes a flow rate sensor (139) operable to sense the flow rate of production (151) flowing through the well surface system (124), after it exits the wellbore (120). The flow rate sensor (139) may include hardware that senses a flow rate of production (121) (Q) passing through the wellhead (130).

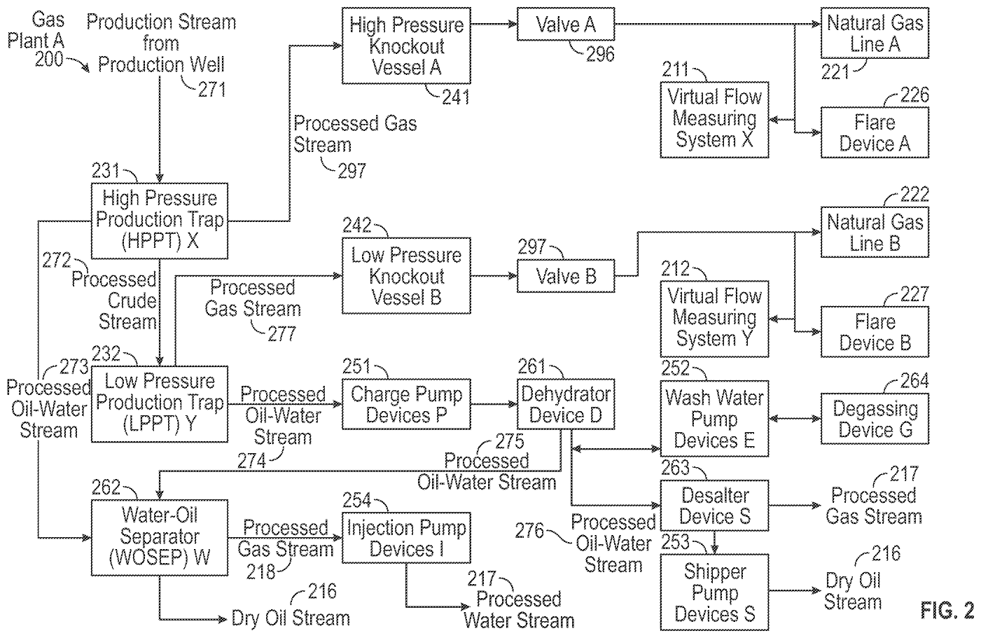

Turning to FIG. 2, FIG. 2 shows a schematic diagram in accordance with one or more embodiments. As shown in FIG. 2, a gas plant (e.g., gas plant A (200)) may include various industrial components for processing production (e.g., production stream from production well (285)) from one or more wells. In some embodiments, a gas plant is a gas-oil separation plant (GOSP) that includes a temporary or permanent facility that separates wellhead fluids into gas components and liquid components, such as dry oil (e.g., dry oil stream (216)) and produced water (e.g., produced water stream (217)). For example, temporary gas-oil separation facilities may correspond to newly drilled wells (e.g., where production potential is being assessed for a drilled well). Likewise, a permanent facility may be coupled to designated pipelines that transport natural gas, dry oil, natural gasoline, liquefied petroleum, condensate, and/or other processed products downstream.

Furthermore, a gas plant may include various production traps (e.g., high pressure production trap (HPPT) X (231), low pressure production trap (LPPT) Y (232)) that include functionality for separating a multi-phase stream (e.g., production stream (271) from production well, processed crude stream (272)) into respective streams (e.g., processed oil-water stream (273), processed oil-water stream (274), processed oil-water stream (275), processed oil-water stream (276), processed gas stream (277), processed gas stream (279)). For example, an HPPT may be a three-phase separator that includes various hardware components, such as a deflector, a water retention baffle, various compartments, etc. When wet crude oil enters an HPPT, the wet crude oil may separate into various outputs, e.g., off-gas, processed wet crude oil (i.e., a wet crude oil output that may still include some water and gas), and oily water (i.e., an oily water output that may include produced water with some remaining crude oil). More specifically, an HPPT may cause a pressure drop among hydrocarbon gases within wet crude oil that results in separation of various chemical components. With respect to an LPPT, an LPPT may be a two-phase separator that receives crude oil from the HPPT and separates remaining gas from the crude oil.

Keeping with FIG. 2, a gas plant may include one or more knockout vessels (e.g., high pressure knockout vessel A (241), low pressure knockout vessel B (242)). For example, a knockout vessel may be a knockout drum (KOD), a knockout trap, a water knockout, or a liquid knockout. With respect to knockout drums, a knockout drum may be a vessel that removes and accumulates various liquids (e.g., condensed liquids and entrained liquids) from relief gases. A knockout drum may have a horizontal configuration or a vertical configuration, which may be determined according to operating parameters and/or gas plant conditions. For example, a horizontal KOD may be used in situations with a large liquid storage capacity and/or high vapor flow in a particular pipeline as well as situations seeking a low pressure drop across the knockout drum. In contrast, a vertical KOD may be used in situations with a low amount of liquid load.

Turning to flare devices, a gas plant may include one or more flare devices (e.g., flare device A (226), flare device B (227)) coupled to a respective knockout drum (e.g., high pressure KOD A (241), low pressure KOD B (242)). For example, raw natural gas may be combusted in an open diffusion flame using one or more flare devices. More specifically, a flare device may include a gas flare, such as a vertical flare stack that includes various flaring equipment, e.g., a flashback prevention section, a pilot flame tip, a spark ignition device, and/or a water seal drum. The flare device may also correspond to a ground-level flare that includes a steel box or cylinder lined with refractory material.

In some embodiments, a flare device may consume raw natural gas, such as associated gas or waste gas. For example, associated gas may be natural gas that is a by-product of oil drilling, which is dominated by methane. Thus, this raw natural gas may be burned because natural gas pipelines are not in place when the oil well is drilled. In some embodiments, a flare device is used to perform routine flaring (also called production flaring) that disposes of unwanted associated gas during crude oil extraction. Besides routine flaring, a flare device may also be used for safety flaring, maintenance flaring, or other types of flaring operations.

A gas plant may include one or more flow conduits, such as a flowline from a well, a pipeline (e.g., for processing crude oil, produced water, and/or various mixture streams), and/or a natural gas line (e.g., natural gas line A (221), natural gas line B (222)) for transporting natural gas away from a gas plant. For example, a flow conduit may include hardware to implement a closed conduit between one or more inlets and one or more outlets. A gas plant may also include various valves (e.g., valve A (296), valve B (297)). A valve may be a closure element with hardware for opening and closing a conduit connection, such as a gate valve, a shutoff valve, a ball valve, a control valve, etc.

In some embodiments, a gas plant may include one or more virtual flow measuring systems (e.g., virtual flow measuring system X (211), virtual flow measuring system Y (212)) that include functionality for determining one or more gas flow rates. In particular, a virtual flow measuring system may include a control system or other computer device that acquires sensor measurements from multiple sensors with respect to a predetermined plant environment. Based on knowledge of this plant environment, a virtual flow measuring system may determine the gas flow rate at a particular location in a gas plant without using a flowmeter or a differential pressure sensor. In some embodiments, for example, a virtual flow measuring system uses a gas flow model to determine a respective flow rate. Examples of gas flow models may include orifice flow equations based on specific gas parameters and/or orifice parameters. Using a gas flow model, a virtual flow measuring system may use pressure data and/or temperature data in relation to a predetermined orifice to determine the gas flow rate.

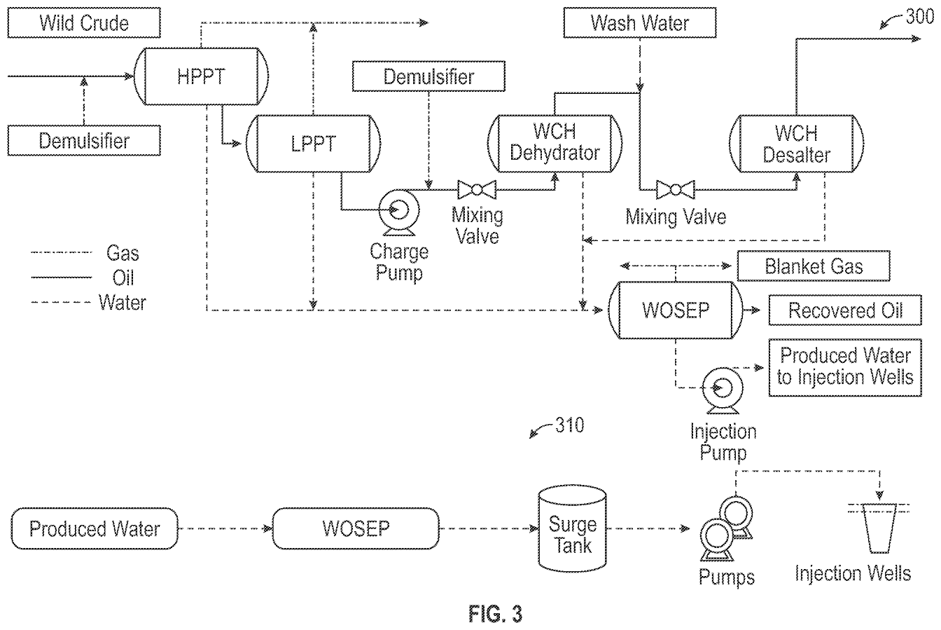

FIG. 3 shows a Gas Oil Separation Plant configuration (300) and associated produced water treatment process (310). The Gas Oil Separation Plant configuration (300) includes one stage dehydration and one stage desalting where a Water Oil Separator (WOSEP) is the single vessel for Deoiling to meet the target. By deploying multi-target time-series based hybrid physics based machine learning model, the Gas Oil Separation Plant can sustain produced water quality in terms of oil in produced water content and ensure product quality to remain within the required targets of 100 ppm or mg/l. Accordingly, plant operators are one-step ahead of the plant operation to anticipate process upsets before actual occurrence.

The main objectives from the multi-target time-series based hybrid physics based machine learning model deployment are listed below:
1. Machine learning techniques to identify root cause analysis for crude quality monitoring
2. Machine learning techniques enhanced by mass balance calculations to sustain crude oil quality specifications
3. Utilize machine learning model coefficients and outputs to generate advisory actionable items
4. Utilize machine learning model augmented with Natural language processing inputs to improve the model
5. Predict quality of crude oil to generate advisory actions for maintaining the crude oil quality within a certain threshold
6. Machine learning techniques to predict future quality of crude oil

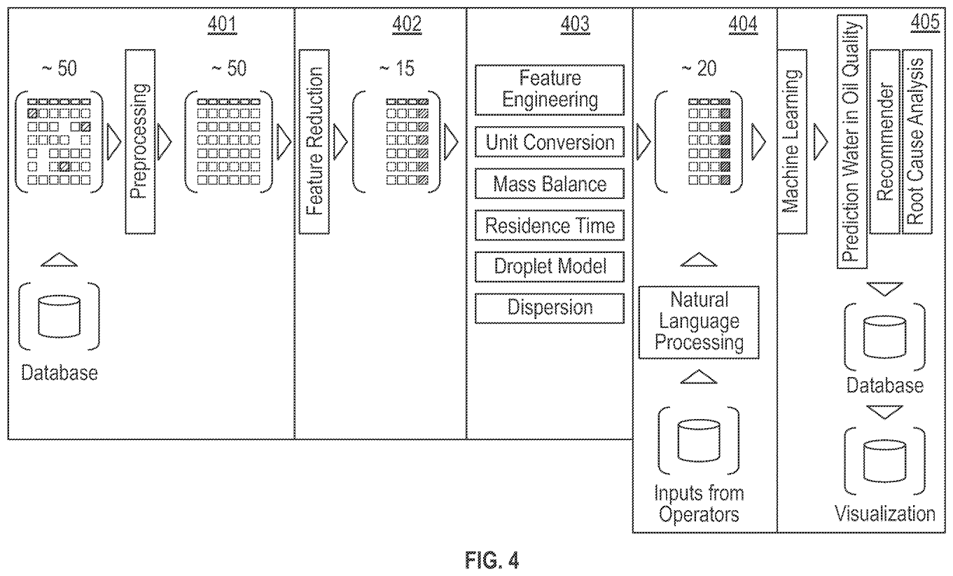

FIG. 4 shows a schematic diagram of five method steps listed below to meet the objectives above.
PREPROCESSING (401)
FEATURE REDUCTION (402)
FEATURE ENGINEERING (403)
NATURAL LANGUAGE PROCESSING (404)
MACHINE LEARNING (405)

Each of the five method steps is described in detail below.

### Preprocessing

The model developed in preprocessing is based on set point values in the oil process train. A setpoint value represents the physical value of each component in the separation process. This model identifies bad actors related to their absolute values and removes outliers.

Main steps taken in preprocessing development are listed below.
1. Identify relevant PI (process intelligence) tags for the separation process, each PI tag corresponds to a time series data storage and holds a single value over time
2. Develop preprocessing of data
3. Set up Machine Learning framework tailored to identifying the crude quality parameters.

Preprocessing of data is needed because a machine learning model requires the data to be numeric and complete, meaning there can be no gaps in the data. The preprocessing performed on the data may be at least the following:
1. Remove outliers. Values more than certain standard deviations away from the mean are removed
2. Transform binary columns to binary values. E.g., whether a pump is on or off.
3. Transform string values to numeric values
4. Ensure everything has the same granularity in time, e.g., a datapoint as small as possible.
5. Remove non-relevant data. It is not necessary to identify a root cause for poor oil-water separation every time a train is shut down. Non-relevant periods are removed from the dataset so that they are not included in the training set.
6. All missing values are replaced by interpolation, except binary values where a forward-fill is performed

### Feature Reduction

A machine learning model using XGBoost (eXtreme Gradient Boosting) with decision trees is used. This algorithm is chosen because it is a recommended solution for multivariate problems where the target variable is non-linearly dependent on multiple time series, which is true for this use case with water-oil separation.

An important attribute of the model is that it accounts for changing production conditions. This is done by finding an optimal training dataset for the current production conditions and using that to train the model.

The output of the model is (1) the estimated oil concentration in the produced water, i.e., the target variable, and (2) the importance of the model's features. The estimated oil content provides a measure of the accuracy of the model. Whenever the estimate is close to the true oil content it is an indication that the trained model has learned the behavior of the separation process, so that the operator can trust the model output. The feature importance is a measure of how important a feature is for the current estimated value. So, when oil concentration is high, the user/operator can understand which factors have contributed most to such a large water concentration. The operator verifies the values measured by lab and online analyzers through reverse engineering and confirms the actual oil content in produced water.

PI tags are used to retrieve archived big data, which is used in this application for data analytics, feature extraction, feature engineering, etc.

The model has access to all the historic trends, and whenever there is an increase in the oil concentration it can look through all of the many PI tags to see which PI tags have been important for achieving this poor water separation and highlight this to the operator.

Most tags relevant to the separation process were identified in feature reduction. A challenge with online analyzers and lab analysis is that multiple sensors represent the same physical value, e.g., three level sensors in a separator. This has two negative consequences: It divides the importance for a separator level between each of the three sensors so that separator level seems to be less important; and it provides the model with “duplicated” data such that training is slower without improving the results.

All tags and their data were sorted for which redundant tags should be removed from the dataset and which redundant tags should be aggregated. Methods implemented for aggregating features are listed below.
Average
Maximum
Minimum
Sum
2 out of 3 then average (accounting for a third PI being out of calibration)

### Feature Engineering

Physics implemented in the model

The following subsections I through III describe the physics that has been implemented into the model through feature engineering.

### I. Mass Balance to Calculate Missing Flow Rates:

The figure below shows all the flow rates in the oil train. The pipes with a flow rate measurement have a flow rate tag associated with the respective flowline in the figure, while the pipes without flow rates have a calculated flow based on mass balance.

To calculate the remaining flow rates the following equation is used:

$$\sum {Q}_{inlet} - \sum {Q}_{outlet} = \sum Δ V / t$$

where
ΣQᵢₙₗₑₜ[m³/s] Sum of all inlet flow rates
ΣQₒᵤₜₗₑₜ [m³/s] Sum of all outlet flow rates
ΣΔV [m³] Sum of all the changes in volume
t [s] Time between data points (granularity)

Changes in volumes are calculated using the same method as for residence time, such that

$$Δ V = {V}_{t 2} - {V}_{t 1}$$

where
V=f(levels, dimensions)

The unknown flow rate is either part of the inlet flow rates, or the outlet flow rates. To calculate each of the missing flow rates the following general equation is used

$${Q}_{?} = \sum {Q}_{i} - \sum {Q}_{o}$$

Where flow rates tags are specified to be either inlet or outlet as in the table below, and outlet flow rates are subtracted from inlet flow rates.

Example calculation—HPPT to LPPT:

At the inlet of the LPPT:

$$\sum {Q}_{1} = {Q}_{?} + A 3 + A 4$$

At the outlet of the LPPT:

$$\sum {Q}_{2} = A 1 + A 2 + Δ V / t$$

This yields

$${Q}_{?} = A 1 + A 2 + Δ V / t - ( A 7 + A 4 )$$

According to the general equation above:

$$\sum {Q}_{i} = A 2 + A 2 + Δ V / t$$

$$\sum {Q}_{o} = A 3 + A 4$$

The following set of rules has been defined to calculate the flow rates marked with “?”:

| Flow rate from | | |
|---|---|---|
| mass balance | inlet | outlet |
| HPPT to LPPT | A1 (LPPT to WOSEP, water) | A3 (recycled crude |
| wet crude | A2 (LPPT to Dehydrator, crude) | from desalter) |
| | LPPT volume change | A4 (water from |
| Manifold to | HPPT to LPPT wet crude | desalter) |
| HPPT wet crude | A5 (HPPT to WOSEP, water) | |
| | HPPT volume | |
| Desalter to | A6 (desalter to LPPT) | A8 (demulsifier |
| desalter | A7 (water outlet desalter to | to desalter) |
| recycled water | transfer pumps) | A9 (wash water |
| | Desalter water volume | to desalter) |

These flow rates have to be calculated in order to calculate residence time for the WOSEP.

### II. Water Concentration Out of LPPT

Assuming all water into dehydrator leaves the dehydrator outlet, it is possible to calculate the water entering the dehydrator from the LPPT:

$$[ water volume from LPPT ] = [ \text{} volume change in dehydrator ] + [ volume water out of dehydrator ]$$

Then, the water concentration in the LPPT is:

$$[ water concentration out of LPPT ] = [ \text{} water volume from LPPT ] / [ total volume from LPPT ]$$

### Where:

[volume change in dehydrator] is based on water level in the dehydrator
[water volume out of dehydrator] and [total volume from LPPT] was calculated using the trapezoidal integral method where the time difference between datapoints is used as dx. The flow rates are measured from sensors

### III. Estimating Water Fraction Out of Dehydrator

Mass balance of dehydrator-desalter:

$${Q}_{i , dehydrator} + {Q}_{i , wash water} = {Q}_{o , crude} + {Q}_{o , disposal}$$

Q_{i,dehydrator} [m³/s] Flow rate of crude out of dehydrator (calculated from total mass balance)
Q_{i,wash water} [m³/s] Flow rate of wash water into desalter (measured)
Q_{o,crude} [m³/s] Flow rate of clean crude out of desalter (measured)
Q_{o,disposal} [m³/s] Flow rate of water out to disposal (measured)

Assuming only water added to the desalter is disposed through water outlet, the following mass balance for oil holds:

$${Q}_{i , dehydrator} ( 1 - {X}_{i , w} ) = {Q}_{o , crude} ( 1 - {X}_{o , w} )$$

X_{i,w} water concentration in flow out of dehydrator (unknown)
X_{o,w} water concentration out of desalter (measured by analyzer)

Solving for X_{i,w}:

$${X}_{i , w} = \frac{( {Q}_{i , dehydrator} - {Q}_{o , crude} ( 1 - {X}_{o , w} ) )}{{Q}_{i , dehydrator}}$$

### Natural Language Processing

In this section of the model, inputs from operator to improve the machine learning model is developed. The below table is developed to provide operator inputs from operator experience.

| | | | Confidence | Operator |
|---|---|---|---|---|
| Vessel | Level | Response | % | Recommendation |
| HPPT | Water level | True/False | XX % | Text |
| | | | | recommendations |
| WOSEP | Water level | True/False | XX % | Text |
| | | | | recommendations |
| WOSEP | Oil level | True/False | XX % | Text |
| | | | | recommendations |
| LPPT | Water level | True/False | XX % | Text |
| | | | | recommendations |
| Dehydrator | Water level | True/False | XX % | Text |
| | | | | recommendations |
| Desalter | Water level | True/False | XX % | Text |
| | | | | recommendations |

The data from the operator is sent through the available commercial Natural language processing tools and these are inputs to the machine learning model for further processing.

### MACHINE LEARNING

Machine learning (ML) model is used to improve the identification of root causes. Combining sensor values, component data sheets, simulated results from a chemical process simulation software (e.g., HYSYS) to calculate physical phenomena important for the oil in water separation process and natural language processing will guide the model to produce better results.

The machine learning model is guided because it does not have to learn the laws of physics through training, instead physically derived features important to the separation process are input directly to the model. Additionally, sensors going into these calculated features can be removed from the training set, which makes training the model faster. The improvement in the model is seen from a better accuracy of the model in terms of estimating oil concentration in produced water when comparing with the actual oil concentration in produced water analyzer. This improvement in accuracy is a sign that the model has learned what is causing poor water separation and thus can identify the root causes.

The root causes identified by the model are the most important factors contributing to the increased water concentration at a given time. Root causes are extracted from the “black box” ML model using Shapley values. It is state of the art within Explainable AI, and implemented in the open source SHAP Python library. SHAP calculates the importance for a set of features X producing the output y when X is input to the model. This is true for any set of features X, which means it is possible to extract local importance's, i.e., importance's at a specific point in time. SHAP also takes the interactions between the features into account, which is a key property for a root cause analysis tool dealing with varying conditions such as in a separation train.

Followings list the Key Process Parameters for a Gas Oil Separation Plants that impact oil water separation and eventually the actual oil concentration in the oil in produced water analyzer.

| Component | Features |
|---|---|
| HPPT | 1. separator-hppt-pressure |
| | 2. separator-hppt-residence_time-water |
| | 3. hppt-demulsifier_concentration |
| | 4. separator-hppt-wosep_and_hppt-flowrate-water |
| LPPT | 1. pump-lppt_charge-dispersion-flowrate |
| | 2. separator-lppt-pressure |
| | 3. separator-lppt-wosep-flowrate-water |
| WOSEP | 1. separator-wosep-disposal-flowrate-water |
| | 2. separator-wosep-level-oil |
| | 3. separator-wosep-level-water |
| Dehydrator | 1. coalescer-dehydrator-dispersion-pressure- |
| | diff_mixing_valve |
| | 2. coalescer-dehydrator-wosep-flowrate-water |
| | 3. corrosion_inhibitor_drum-dehydrator- |
| | flowrate-chemical |
| | 4. dehydrator-demulsifier_concentration |
| | 5. coalescer-dehydrator-level-water |
| Desalter | 1. coalescer-desalter-dispersion-pressure- |
| | diff_mixing_valve |
| | 2. coalescer-desalter-wosep-flowrate-water |
| | 3. wash_water_drum-desalter-flowrate-water |
| | 4. coalescer-desalter-level-water |

Process Parameters for a Gas Oil Separation Plants impact oil water separation and eventually the actual oil concentration in the oil in water analyzer. The results from the models are shown in FIGS. 5A and 5B.

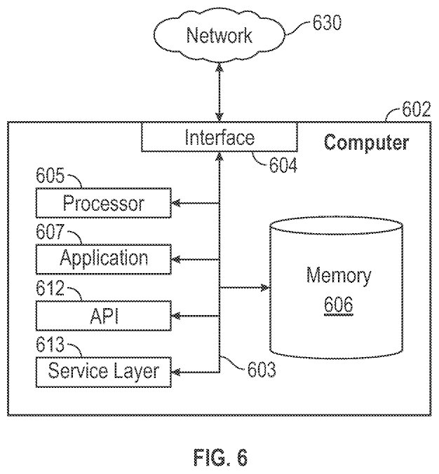

Embodiments may be implemented on a computer system. FIG. 6 is a block diagram of a computer system (602) used to provide computational functionalities associated with described algorithms, methods, functions, processes, flows, and procedures as described in the instant disclosure, according to an implementation. The illustrated computer (602) is intended to encompass any computing device such as a high performance computing (HPC) device, a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer (602) may include a computer that includes an input device, such as a keypad, keyboard, touch screen, or other device that can accept user information, and an output device that conveys information associated with the operation of the computer (602), including digital data, visual, or audio information (or a combination of information), or a GUI.

The computer (602) can serve in a role as a client, network component, a server, a database or other persistency, or any other component (or a combination of roles) of a computer system for performing the subject matter described in the instant disclosure. The illustrated computer (602) is communicably coupled with a network (630). In some implementations, one or more components of the computer (602) may be configured to operate within environments, including cloud-computing-based, local, global, or other environment (or a combination of environments).

At a high level, the computer (602) is an electronic computing device operable to receive, transmit, process, store, or manage data and information associated with the described subject matter. According to some implementations, the computer (602) may also include or be communicably coupled with an application server, e-mail server, web server, caching server, streaming data server, business intelligence (BI) server, or other server (or a combination of servers).

The computer (602) can receive requests over network (630) from a client application (for example, executing on another computer (602)) and responding to the received requests by processing the said requests in an appropriate software application. In addition, requests may also be sent to the computer (602) from internal users (for example, from a command console or by other appropriate access method), external or third-parties, other automated applications, as well as any other appropriate entities, individuals, systems, or computers.

Each of the components of the computer (602) can communicate using a system bus (603). In some implementations, any or all of the components of the computer (602), both hardware or software (or a combination of hardware and software), may interface with each other or the interface (604) (or a combination of both) over the system bus (603) using an application programming interface (API) (612) or a service layer (613) (or a combination of the API (612) and service layer (613). The API (612) may include specifications for routines, data structures, and object classes. The API (612) may be either computer-language independent or dependent and refer to a complete interface, a single function, or even a set of APIs. The service layer (613) provides software services to the computer (602) or other components (whether or not illustrated) that are communicably coupled to the computer (602). The functionality of the computer (602) may be accessible for all service consumers using this service layer. Software services, such as those provided by the service layer (613), provide reusable, defined business functionalities through a defined interface. For example, the interface may be software written in JAVA, C++, or other suitable language providing data in extensible markup language (XML) format or other suitable formats. While illustrated as an integrated component of the computer (602), alternative implementations may illustrate the API (612) or the service layer (613) as stand-alone components in relation to other components of the computer (602) or other components (whether or not illustrated) that are communicably coupled to the computer (602). Moreover, any or all parts of the API (612) or the service layer (613) may be implemented as child or sub-modules of another software module, enterprise application, or hardware module without departing from the scope of this disclosure.

The computer (602) includes an interface (604). Although illustrated as a single interface (604) in FIG. 6, two or more interfaces (604) may be used according to particular needs, desires, or particular implementations of the computer (602). The interface (604) is used by the computer (602) for communicating with other systems in a distributed environment that are connected to the network (630). Generally, the interface (604) includes logic encoded in software or hardware (or a combination of software and hardware) and operable to communicate with the network (630). More specifically, the interface (604) may include software supporting one or more communication protocols associated with communications such that the network (630) or interface's hardware is operable to communicate physical signals within and outside of the illustrated computer (602).

The computer (602) includes at least one computer processor (605). Although illustrated as a single computer processor (605) in FIG. 6, two or more processors may be used according to particular needs, desires, or particular implementations of the computer (602). Generally, the computer processor (605) executes instructions and manipulates data to perform the operations of the computer (602) and any algorithms, methods, functions, processes, flows, and procedures as described in the instant disclosure.

The computer (602) also includes a memory (606) that holds data for the computer (602) or other components (or a combination of both) that can be connected to the network (630). For example, memory (606) can be a database storing data consistent with this disclosure. In one or more embodiments, the memory (606) may be a non-transitory computer-readable storage medium. For example, the memory (606) stores a method to perform oil in produced water analysis consistent with this disclosure. Although illustrated as a single memory (606) in FIG. 6, two or more memories may be used according to particular needs, desires, or particular implementations of the computer (602) and the described functionality. While memory (606) is illustrated as an integral component of the computer (602), in alternative implementations, memory (606) can be external to the computer (602).

The application (607) is an algorithmic software engine providing functionality according to particular needs, desires, or particular implementations of the computer (602), particularly with respect to functionality described in this disclosure. For example, application (607) can serve as one or more components, modules, applications, etc. Further, although illustrated as a single application (607), the application (607) may be implemented as multiple applications (607) on the computer (602). In addition, although illustrated as integral to the computer (602), in alternative implementations, the application (607) can be external to the computer (602).

There may be any number of computers (602) associated with, or external to, a computer system containing computer (602), each computer (602) communicating over network (630). Further, the term “client,” “user,” and other appropriate terminology may be used interchangeably as appropriate without departing from the scope of this disclosure. Moreover, this disclosure contemplates that many users may use one computer (602), or that one user may use multiple computers (602).

In some embodiments, the computer (602) is implemented as part of a cloud computing system. For example, a cloud computing system may include one or more remote servers along with various other cloud components, such as cloud storage units and edge servers. In particular, a cloud computing system may perform one or more computing operations without direct active management by a user device or local computer system. As such, a cloud computing system may have different functions distributed over multiple locations from a central server, which may be performed using one or more Internet connections. More specifically, cloud computing system may operate according to one or more service models, such as infrastructure as a service (IaaS), platform as a service (PaaS), software as a service (SaaS), mobile “backend” as a service (MBaaS), serverless computing, artificial intelligence (AI) as a service (AIaaS), and/or function as a service (FaaS).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, any means-plus-function clauses are intended to cover the structures described herein as performing the recited function(s) and equivalents of those structures. Similarly, any step-plus-function clauses in the claims are intended to cover the acts described here as performing the recited function(s) and equivalents of those acts. It is the express intention of the applicant not to invoke 35 U.S.C. § 112(f) for any limitations of any of the claims herein, except for those in which the claim expressly uses the words “means for” or “step for” together with an associated function.

What is claimed is:

1. A method to perform oil in produced water analysis, comprising:
generating a time-series and physics based machine learning model of a gas oil separation plant;
generating, based on machine learning model coefficients and outputs of soft sensors, advisory actionable items for maintaining a crude oil quality within a pre-determined threshold; and
presenting, to a user, the advisory actionable items,
wherein generating the time-series and physics based machine learning model comprises:
performing feature engineering based on one or more of
calculating missing flow rates using mass balance,
calculating water concentration of output from a low pressure production trap (LPPT) of the gas oil separation plant, and
estimating water fraction out of a dehydrator of the gas oil separation plant, and
augmenting the time-series and physics based machine learning model with natural language processing inputs from the user.

2. The method according to claim 1 further comprising preprocessing and feature reduction.

3. The method according to claim 2, wherein the preprocessing comprises identifying process intelligence (PI) tags corresponding to a time series data and a single value over time, preprocessing data, and setting up a machine learning frame work to identify crude quality parameters.

4. The method according to claim 3, wherein preprocessing data comprises removing outliers, transforming binary to binary values, transforming string values to numeric values, removing non-relevant data, and interpolating missing values.

5. The method according to claim 2, wherein the feature reduction comprises using the PI tags to retrieve archived big data.

6. The method according to claim 5, wherein the PI tags are sorted to remove and aggregate redundant tags.

7. The method according to claim 1, wherein the advisory actionable items identify root causes for poor water separation.

8. A non-transitory computer readable medium storing instructions executable by a computer processor, the instructions comprising functionality for:
generating a time-series and physics based machine learning model of a gas oil separation plant;
generating, based on machine learning model coefficients and outputs of soft sensors, advisory actionable items for maintaining a crude oil quality within a pre-determined threshold; and
presenting, to a user, the advisory actionable items,
wherein generating the time-series and physics based machine learning model comprises:
performing feature engineering based on one or more of
calculating missing flow rates using mass balance,
calculating water concentration of output from a low pressure production trap (LPPT) of the gas oil separation plant, and
estimating water fraction out of a dehydrator of the gas oil separation plant, and
augmenting the time-series and physics based machine learning model with natural language processing inputs from the user.

9. The non-transitory computer readable medium according to claim 8 further comprising preprocessing and feature reduction.

10. The non-transitory computer readable medium according to claim 9, wherein the preprocessing comprises identifying process intelligence (PI) tags corresponding to a time series data and a single value over time, preprocessing data, and setting up a machine learning frame work to identify crude quality parameters.

11. The non-transitory computer readable medium according to claim 10, wherein preprocessing data comprises removing outliers, transforming binary to binary values, transforming string values to numeric values, removing non-relevant data, and interpolating missing values.

12. The non-transitory computer readable medium according to claim 9, wherein the feature reduction comprises using the PI tags to retrieve archived big data.

13. The non-transitory computer readable medium according to claim 12, wherein the PI tags are sorted to remove and aggregate redundant tags.

14. The non-transitory computer readable medium according to claim 8, wherein the advisory actionable items identify root causes for poor water separation.
